(19) United States
(12) Patent Application Publication
(10) Pub. No.: US 2007/0098822 A1
(43) Pub. Date: May 3, 2007

(54) Food compositions and methods

(76) Inventor: Roy J. Mankovitz, Montecito, CA (US)

Correspondence Address: Julie K. Staple;Gifford, Krass, Groh, Sprinkle, Anderson & Citkowski, P.C., PO Box 7021, Troy, MI, 48007-7021, US

(21) Appl. No.: 11/414,863

(22) Filed: May 1, 2006

Related U.S. Application Data
(60) Provisional application No. 60/731,762, filed on Oct. 31, 2005.

(51) Int. Cl. A61K 36/899; A23K 1/00; A23K 1/18

(52) U.S. Cl. 424/750; 426/2; 426/635

(57) ABSTRACT

Compositions are provided according to the present invention which include microorganisms and/or products of microorganismal metabolism. Humans and other animals have difficulty gaining nutritional benefit from many highly abundant plant materials, such as cellulose. A traditional means of gaining the benefit of these nutrients has been through the cultivation of animals capable of utilizing such materials, subsequently consuming their meat and milk products. However, raising such animals is time consuming and expensive. Compositions and methods according to the present invention allow circumvention of the use of these cultivated animals for meat and milk. Microorganisms are isolated from an animal which is a traditional source of food for a second type of animal, such as humans or pets, in order to produce compositions of the invention. Such compositions provide nutritional benefit to the consumer.

### REFERENCE TO RELATED APPLICATIONS

This application claims priority to U.S. Provisional Patent Application Ser. Nos. 60/696,658, filed Jul. 5, 2005, and 60/731,762, filed Oct. 31, 2005, the entire content of both of which are incorporated herein by reference.

### FIELD OF THE INVENTION

The present invention generally relates to food compositions and processes for their manufacture. In one specific embodiment, the present invention relates to food compositions including microorganisms isolated from the digestive system of an animal which is a traditional food source for a type of animal which is an intended recipient of an inventive composition.

### BACKGROUND OF THE INVENTION

While modern science has elucidated many biological processes at the cellular and even molecular level, the interactions between microbial organisms and mammalian organisms have been largely uncharacterized, although there is considerable evidence of their importance.

In particular, it is well established that various types of microbes ordinarily live in the mammalian gut. Such microbes, termed intestinal flora, are known to have effects on the organism that they colonize. For example, some bacteria synthesize and make certain essential nutrients available to the host animal. In humans, for instance, vitamin K is an essential compound which may be provided by bacterial synthesis in the gut.

A role for microorganisms in digestion has been extensively studied in some animals, such as ruminants. In other species exact functions of digestive system microorganisms are less understood.

The gastrointestinal system varies between species but generally includes several different sections having specific functions in the digestive process of the animal. In particular, digestive systems are configured differently depending on the usual food source of the animal. For example, a typical carnivore digestive system is configured to digest protein efficiently along with fats and some carbohydrates. A carnivore system is characterized by anatomical structures functional to mechanically dissociate food, such as teeth, a single acid secreting stomach which includes acid activated enzymes functional to break down proteins, the small intestine for further digestion and absorption of the food, and the large intestine which also functions to absorb some nutrients. Microorganisms present in regions of the carnivore digestive system function to digest some substrates indigestible by the carnivore, as well as provide some essential nutrients.

In contrast, an herbivore gastrointestinal system is configured to utilize carbohydrates derived from plants. In this regard, herbivores include ruminants, a type of animal that has a specialized multigastric configuration of the digestive system, and non-ruminant herbivores. Ruminants are characterized by a multigastric system having 3 to 4 compartments, including the rumen, reticulum, omasum and abomasum. The multigastric configuration functions to allow repetitive mechanical breakdown of plant material and provides an environment conducive to fermentation of the plant material by resident microorganisms.

In addition to a role in digestive metabolism, microorganisms are believed to play a more general role in the health of host animals. A number of diseases and disorders are believed to be related to alterations of number and/or types of microorganisms represented in the intestinal flora. For example inflammatory bowel diseases, such as Crohn's disease and ulcerative colitis are associated with reduced diversity of intestinal flora. (Ott, S. J. et al., Reduction in diversity of the colonic mucosa associated bacterial microflora in patients with active inflammatory bowel disease. Gut, 53:685-693, 2004.) Further, modern “lifestyle” disorders such as cancer, heart disease, hypertension, diabetes, senile dementia, microbial or viral infection, auto-immune disorder, atopic dermatitis, as well as various allergies and food sensitivities, more prevalent in recent history, are thought to be associated with changes in intestinal flora.

Both human and cultivated animal diets have changed significantly in recent history. Modem humans and the animals they raise for food or as companions now consume highly processed foods and/or foods never or rarely consumed in the natural or primitive environment. Further, the advent of high volume food manufacturing and relatively inexpensive snack foods has contributed to changes in the overall composition of foods included in a modem diet compared to previous eras. For instance, populations of modem humans eat more simple carbohydrates than were available historically. A cultivated animal's diet is now constructed according to convenience and to promote fast growth, rarely providing the foods the animal would eat in the natural state or as cultivated in a primitive society. A companion animal's diet is sanitized and largely adapted to human concepts of a pet's food preferences. These relatively recent changes are believed to cause distortions of the intestinal flora in humans and animals exposed to modem habits since modem diets support different populations of microorganisms than a traditional or primitive diet based on natural foods.

In addition to changes in diet, both humans and cultivated animals are routinely exposed to antibiotics which affect not only pathogenic microorganisms but benign and beneficial microorganisms as well. The systemic treatment of an individual during a course of antibiotics may result in elimination of gut microorganisms, many varieties of which may not be replaced if exposure to microorganisms is limited.

The diversity of modem intestinal flora in humans and other animals is believed to be limited by the paucity of sources of potential exposure to microorganisms. Currently human and even animal hygiene standards are at their historical zenith, with both desirable and unanticipated less desirable results. There is evidence that limited exposure of humans to dirt, dust, animals, and the various antigens found therein, such as bacteria and viruses, can predispose an individual to immune disorders, such as allergies and asthma.

Current dietary preferences and/or habits based on available food products also have a role in limiting exposure of modern humans and other animals to microorganisms. In particular, modem humans who eat meat typically prefer the muscle meat of an animal rather than the organ meat. In contrast, earlier societies valued all parts of the body of a source animal, including internal organs such as the heart, liver, kidneys, and, importantly, the digestive system including the tongue, the stomach, intestines and intestinal contents. For example, an account of a traditional Native American diet describes the use of buffalo entrails as food including intestines “full of half-fermented, half-digested grass and herbs . . . ” (John Lame Deer & Richard Erdoes, Lame Deer, Seeker of Visions, p. 122, Simon & Schuster, 1972) Further, both cultivated and wild animals which are sources of nutrition for humans and pets historically had access to food likely to expose the animals to microorganisms, such as pasture grass and other wild growing plants which were not processed to remove or inhibit microorganisms.

In addition, the distorted diet of the modern human leads to disorders due to nutritional deficiencies and/or over-exposure to particular foods. For instance, certain conditions, such as cardiovascular disease, adult-onset diabetes and obesity are associated with a modern lifestyle, including fast food and highly processed foods. High volume food manufacturing creates such unnatural foods at relatively low prices, such that natural foods are available only at premium prices. This creates the situation in which low-income populations eat disproportionate amounts of manufactured unnatural foods and suffer disproportionately from associated disease.

Ironically, the natural environment is filled with nutrients unavailable to humans and many other animals due to an inability to digest many plant materials, such as cellulose. A traditional means of gaining the benefit of these nutrients has been through the cultivation of animals capable of utilizing such materials. As outlined above, ruminants in particular are able to gain nutritious benefit from plant materials through a cooperative arrangement with intestinal flora. The intestinal microorganisms have the ability to ferment plant materials to provide for their own growth and, in addition, produce materials which provide a nutritional benefit to the host organism.

However, the time and energy spent raising such animals makes their meat and milk products expensive. Further, efforts to decrease the cost of animal food products have led to animal feed which is non-natural, negatively affecting the meat and milk products.

Large amounts of plant material normally cannot be consumed by humans and certain pet animals due to the high level of cellulose. Plant materials, such as grass, are plentiful and could serve as a nutritious and health promoting food, if they could be adapted for human and animal digestion. By producing such products on a mass scale from such plentiful and widely available raw materials that have heretofore been considered unfit for consumption, nutritious foods can be cheaply and easily provided, for instance, to large masses of undernourished peoples around the globe, mitigating the loss of lives from starvation.

Thus, in view of the disorders associated with distortion of diversity of intestinal flora and there is a continuing need for compositions and methods designed to provide exposure to microorganisms in order to promote health of humans and other animals. In addition, current unavailability of nutritional content of many plants makes it highly desirable to produce compositions and methods for making these nutrients available.

### SUMMARY OF THE INVENTION

A food composition is provided by the present invention which includes a digestion or fermentation product of a food component of the natural diet of a source animal. The source animal is a traditional food source of a second type of animal. The food component is preferably a food found in the native habitat of the source animal. In one embodiment, a food component includes a grass.

A food composition according to the present invention may further include microorganisms isolated from a source animal, a digestive enzyme typically found in the digestive system of the source animal and/or a non-enzymatic secretion of the digestive system of the source animal.

A food composition intended for consumption by humans is provided according to the present invention. In such an embodiment, the source animal is a traditional food source for a human. An example of such a source animal is a weaned ruminant.

Further provided is a food composition intended for consumption by an animal commonly kept as a household pet. Source animals which are traditional foods for cats or dogs include ruminants, pigs, poultry, birds, fish, rodents, rabbits, hares, reptiles and amphibians.

A process for producing a food composition is provided which includes contacting one or more foods typically consumed as a part of a natural diet of a source animal and a plurality of microorganisms isolated from the source animal to produce a mixture. The mixture is incubated under conditions suitable for reaction of the mixture to produce a product such as a fermentation product of the one or more foods, a digestion product of the one or more foods, or a combination of these.

Further included in an inventive process is a step of adding a component typically found in the digestive system of the source animal to the mixture. Such a component is illustratively an enzyme produced by a cell of the digestive system of the source animal, non-enzymatic digestive secretion produced by a cell of the digestive system of the source animal, or a combination of these.

Further provided by the present invention is a composition which includes a food component of the natural diet of a source animal having a native habitat, wherein the source animal is a traditional food source of a second type of animal; a plurality of microorganisms isolated from a source animal; and a digestion or fermentation product of the food component.

In a preferred example, the food component is a grass. Also preferred is an embodiment in which the source animal is a weaned ruminant

### DETAILED DESCRIPTION OF THE INVENTION

Food compositions, as well as methods of generating them and using them, are provided according to the present invention.

Food Compositions

Compositions are provided which include microorganisms and/or which are generated using microorganisms.

Microorganisms for use in food compositions and related methods are isolated from the digestive system of an animal which is a traditional food source for a second type of animal. Such isolated microorganisms may be used directly in a food composition and/or related method, and may also be cultured and/or amplified for such use.

The animal from which microorganisms are obtained is called a “source animal” herein, to indicate both that this animal is a source of microorganisms included in an inventive composition and that the animal is a traditional food source for a second type of animal which is an intended recipient of an inventive composition as described in more detail below. The terms “second type of animal” and “individual of a second type of animal” as used herein refer to an intended recipient of an inventive composition.

The term “traditional food source” as used herein is intended to mean an animal eaten for nutritive purposes in a natural setting by a second type of animal. Thus, for example, any of various herbivores are a traditional food source for any of various carnivores or omnivores. In contrast however, a carnivore is not considered a traditional food source for an herbivore.

Illustratively, microorganisms are isolated from the digestive system of a ruminant. Ruminants are herbivores which are a traditional source of food for a number of other animals, especially humans, but also including other relatively large carnivores and/or omnivores. Ruminants include cattle, sheep, goats, bison, buffalo, deer, elk, antelope, moose, and llamas for instance.

In other examples, microorganisms are isolated from the digestive system of an animal such as a pig, a poultry animal such as a chicken or a turkey, a bird, including a game bird, a fish, a shellfish, a horse, a rodent, a rabbit, and a hare. Such animals are a traditional source of food for humans and other relatively large carnivores and/or omnivores.

In a further example, microorganisms are isolated from the digestive system of an animal such as pigs, poultry, birds, fish, rodents, rabbits, hares, small reptiles and amphibians which are traditional food sources for smaller carnivores and/or omnivores such as domesticated dogs and cats. In addition, some ruminants described above are a traditional source of food for dogs. For instance, dogs may kill and eat cattle, sheep, goats, bison, buffalo, deer, elk, antelope, moose, and/or llamas.

A source animal is preferably raised in an environment as similar as possible to the environment in which the species historically lived prior to domestication. Thus a preferred source animal has never been exposed to exogenously administered growth hormones, antibiotics, pesticides, or other drugs.

It is highly preferred that the source animal is an animal fed a “natural” diet” over the span of its life. Cultivated ruminants are currently fed a distorted diet in order to promote maximum growth. For example, a typical feed preparation for a growing cow may contain about 20% grain and 80% silage or other roughage such as hay. In the final stage of preparing the cow for market, it may be fed a “finishing” diet including about 80% grain or more. Such a feeding regimen includes a disproportionate amount of grain compared to the diet a foraging animal in an uncultivated pasture would consume. Further, it is believed that high grain content in a food source animal's diet results in changes in composition of the food products produced from the animal. For instance, it has been shown that grain fed beef can have a higher amount of saturated fatty acids and an unfavorable ratio of saturated fatty acids to unsaturated fatty acids. P. French et al., Fatty acid composition, including conjugated linoleic acid, of intramuscular fat from steers offered grazed grass, grass silage, or concentrate-based diets, J. Anim. Sci., 2000, 78:2849-2855. Thus, it is particularly preferred to isolate microorganisms from an animal fed on a “natural” diet.

The term “natural diet” as used herein is intended to mean that the source animal is fed food growing wild in the animal's native habitat, and which excludes foods not normally found in such a native habitat.

The term “native habitat” is intended to preferentially include the habitat of the breed of a source animal prior to human domestication. By way of example, but not limitation, cattle would not naturally be found in habitats that receive large amounts of snow, since they have no way to reach grass under the snow in winter.

Optionally, a source animal may be an animal whose native habitat is located on the continent of Africa. In particular, a source animal may be an animal whose native habitat is located in the tropical region of Africa, that is, the region on either side of the equator extending between two parallels of latitude on the earth, one 23°27′ north of the equator and the other 23°27′ south of the equator.

A source animal may be a carnivore, herbivore or omnivore.

In a particular example, a source animal having an African native habitat is an herbivore of the order Artiodactyla. Common names for African animals of this type include topi, hartebeest, wildebeest, waterbuck, gerenuk, gemsbok, impala, gnu, gazelle, giraffe, okapi, kudu and eland. A source animal of particular interest may be the African cape buffalo (Syncerus caffer). These and other African and non-African animals may be a source animal, including those animals described in standard references such as: Jones, C. 1984. Tubulidentates, Proboscideans, and Hyracoideans, in Orders and Families of Recent Mammals of the World. Edited by Anderson, S. and J. K. Jones, Jr. John Wiley and Sons, N.Y. pp. 523-535; Nowak, R. M. 1991. Walker's Mammals of the World. Fifth Edition. Johns Hopkins University Press, Baltimore; Thenius, E. 1990. Even-toed Ungulates. In Grzimek's Encyclopedia of Mammals. Volume 5. Edited by Parker, S. P. New York: McGraw-Hill. Pp. 1-15; and Webb, J. E., J. A. Wallwork, and J. H. Elgood. 1979, Guide to Living Mammals, Second Edition. Bell and Blain Ltd., Glasgow.

In this context it is to be understood that a source animal is preferably an animal that has been weaned. An unweaned animal typically has a different set of microorganisms in the gut since the animal has not yet been exposed to many of the typical sources of gut flora. Thus, milk, milk products, and milk components, such as whey, are not among foods considered “natural” for a weaned source animal.

The components of a natural diet will depend on the source animal. Animals in the wild will select a diet which they are adapted to digest and which corresponds to their usual food seeking behavior.

Common food seeking behavior of some animals includes “grazing” and “browsing”. For example, wild grazers will eat a diet composed primarily of grasses and other ground plants such as clover. Grazers include cattle and bison among others. Wild browsers will eat grasses and ground plants, and in addition, will eat leaves and small twigs from trees and bushes. Browsing animals include deer and goats among others.

Exemplary African source animals also display various food selection preferences. For instance browsers include such source animals as the giraffe and Guenther's dik-dik and grass or ground plant preferring animals include the hartebeest and wildebeest.

The diets of herbivores also contain other material ingested along with grasses and ground plants, such as small amounts of seeds and insects.

In addition to larger herbivores discussed above, smaller herbivores, such as rabbits and hares are considered source animals for humans and certain pets, including cats and dogs. The natural diet of such herbivores includes grasses and ground plants.

Rodents such as mice, rats and squirrels are typically natural omnivores, eating a natural diet they will consume such foods as insects, terrestrial non-insect arthropods, leaves, roots and tubers, wood, bark, stems, grains, nuts, fruit, seeds, fungi, young birds, eggs, amphibians and reptiles. Among rodents, rats are known to each nearly anything edible as part of a natural diet including birds, mammals, amphibians, reptiles, fish, eggs, carrion, insects, terrestrial non-insect arthropods, mollusks, terrestrial worms, aquatic crustaceans, echinoderms, other marine invertebrates, zooplankton, and fungus.

Small birds eating a natural diet consume such foods as insects, seeds, buds, berries, fruit, flower nectar, cereals, grain, and grass.

Poultry, including domestic or wild chickens, turkeys, guinea fowl, pheasants, quail, pigeons, doves and peacocks, are typically omnivores whose natural diet includes fruits, seeds, leaves, shoots, flowers, tubers, roots, arthropods, snails, worms, lizards, snakes, small rodents, avian nestlings and eggs, for example. Poultry also include aquatic birds such as ducks and geese, which are typically herbivores whose natural diet includes such foods as vegetation, including leaves, roots and tubers, seeds, grains, nuts and algae. These animals are also occasional omnivores whose natural diet may include worms, gastropods, arthropods, and small fish.

Pigs include members of the family Suidae. Pigs are typically omnivores whose natural diet includes bulbs, carrion, earthworms, eggs, fruit, fungi, leaves, roots, tubers, snails, and small vertebrates such as nesting birds and small rodents.

Small reptiles include skinks and lizards which are generally insectivorous, eating spiders, millipedes, crickets, termites, grasshoppers, caterpillars, non-insect arthropods, beetles, and beetle larvae; and snakes which eat small birds, small mammals, amphibians, fish, insects, terrestrial non-insect arthropods, mollusks, and terrestrial worms.

Amphibians, such as frogs and toads consume a natural diet including insects, annelids and gastropods.

The natural diet of fish includes fish, fish eggs, aquatic vegetation, and aquatic invertebrates such as plankton, brine shrimp, and krill.

A source animal may be bred and/or maintained as a cultivated animal by humans in order to obtain microorganisms and/or other contents of the gastrointestinal tract. Optionally, a source animal is caught in its native habitat, a sample of microorganisms and/or other contents of the gastrointestinal tract obtained for use in an inventive composition and/or amplification for use in an inventive composition. The animal may then be returned to the wild. In a further option, a source animal is caught in its native habitat and then maintained in captivity. Where a source animal is cultivated and/or maintained in captivity, it is fed a natural diet.

Grasses eaten as part of a natural diet include those of the family of “true grasses”, that is, those classified in the family Poaceae (also known as Graminae). There are about 700 genera and nearly 12,000 species of grasses. Such grasses generally have hollow stems with nodes at intervals in the stems where leaves may be located. The fruit of such grasses is known as a grain. The family Fabaceae also includes a number of plants found in the natural diet of herbivores including clover and alfalfa.

Some of the grasses in the family Poaceae are mass cultivated as food and are known as cereals, including maize (or corn), wheat, oats, rye, rice, and barley. It is these and similar cultivated cereal grains which are typically included in disproportionate amounts in the modern diet of a food source animal compared to a natural diet. It is particularly preferred that the animal is fed a diet of natural foods in the proportion that the animal would feed on in a natural diet. Thus, since cereal grain is relatively rare in the wild habitat, a grazing herbivore, such as a buffalo or cow, would have little cereal grain in its natural diet. An herbivore source animal from which microorganisms are isolated is therefore preferably an animal fed predominantly grass with little or no cereal grain. For instance, a preferred diet includes less than 5% cereal grain, and preferably less than 2% cereal grain. Highly preferred is a source animal fed substantially no cereal grain.

In addition to the composition of the source animal's diet, the quality of food consumed by a source animal is considered important as well since this can influence the number and identity of microorganisms present in the gut. A preferred source animal is one fed a diet of organically gown food throughout its life, that is, food which is minimally processed, not genetically modified, grown without pesticides and herbicides, and grown using only natural fertilizer if any is used.

Microorganisms and Isolation of Microorganisms

An isolated sample of microorganisms may be obtained from any of various regions of the digestive system of the source animal. For example, microorganisms may be isolated from the mouth, the esophagus, the pharynx, the stomach, the rumen, the omasum, the abomasum, the reticulum, the small intestine, the large intestine, the caecum, or combinations of these.

In one embodiment, the contents of a portion of the digestive system are obtained and a sample of microorganisms is isolated from the contents. For example, contents of the digestive system of an animal include ingested food particles, partially digested material and fecal material. In a further embodiment, microorganisms may be isolated from a digestive system tissue. Thus, for example, scrapings from the walls of the digestive system are one type of sample of microorganisms from a digestive system tissue. Optionally, an isolated sample of microorganisms obtained from the digestive system of a source animal may be combined with isolated samples from other animals.

In a further embodiment a sample of microorganisms is obtained from the digestive system of the source animal and amplified by growing the microorganisms on a culture medium to yield an amplified microorganism culture.

In a highly preferred embodiment the culture medium includes one or more foods traditionally consumed as a natural diet by the type of animal from which the microorganisms are obtained.

Thus, for example where the sample is obtained from the digestive system of an herbivore, a culture medium includes a grass and/or ground plant, such as clover, or an extract thereof. A grass included as a culture medium is preferably an organically grown and minimally processed natural grass of a type that would be found in the animal's native habitat. Grasses which may be included in a culture medium include those of the family of “true grasses”, that is, those classified in the family Poaceae (also known as Graminae). Another exemplary component is a plant from the family Fabaceae, such as a clover and/or alfalfa, which may also be included in a culture medium for microorganisms. However, a culture medium preferably includes little or no cereal grain from the grass family. For instance, a preferred culture medium contains less than 5% of a cereal grain and further preferably contains less than 2%. Highly preferred is a culture medium which contains substantially no cereal grain. Further, since microorganisms are obtained from weaned animals, a culture medium contains substantially no milk, milk products or milk components.

In an example where a source animal is a carnivore or omnivore, a culture medium includes typical contents of such an animal's digestive system and particularly, includes components of the animal's natural diet.

Techniques and other culture media and components thereof for amplification of microorganisms from a sample obtained from the digestive system of an animal are exemplified in J. P. Salanitro et al., Bacteria isolated from the duodenum, ileum, and cecum of young chicks, Appl. Environ. Microbiol. 35(4):782-790, 1978; W. E. C. Moore and L. V. Holdeman, Human Fecal Flora: The Normal Flora of 20 Japanese-Hawaiians, Appl. Microbiol. 27(5):961-979, 1974; M. Morotomi et al., Distribution of indigenous bacteria in the digestive tract of conventional and gnotobiotic rats, Infect. Immun. 11(5):962-968, 1975; P. Quinn, Clinical Veterinary Microbiology, Mosby, 1994; and R. M. Atlas, Handbook of Microbiological Media, CRC Press; 3rd ed., 2004.

In one embodiment a sample obtained from a source animal is tested prior to culture to determine the number and diversity of microorganisms present. For example, a portion of the sample may be subjected to cell or molecular analysis, such as polymerase chain reaction (PCR) analysis, to characterize the microorganisms present. Following obtention of an amplified microorganism culture, cell or molecular analysis, such as a PCR analysis, may be performed to determine the diversity of the amplified culture. Comparison of first and second PCR analyses may be performed to ascertain the number and diversity of microorganism species present in the sample and the amplified culture. This information may be used, for instance, to modify culture conditions to achieve a greater diversity in the amplified culture.

Cell analysis of a sample of microorganisms may include standard microbiological analysis, for instance growing a sample on a selective medium, microscopic examination, and/or staining. In addition other molecular techniques are applicable in analysis of microorganisms, such as isolation of nucleic acids and Southern or Northern blotting.

Cell and molecular analysis of microorganism samples and culture samples may be performed according to standard techniques. Exemplary protocols and conditions for PCR and other analyses of gut microorganisms are set forth in references such as J. Sambrook et al., Molecular Cloning: A Laboratory Manual, Cold Spring Harbor Laboratory Press; 3rd ed., 2001; Eckburg, P. B., et al., Diversity of the Human Intestinal Microbial Flora, Science. 308: 1635-1638, 2005; Nordgard, L. et al., Nucleic Acid isolation from ecological samples-vertebrate gut flora, Methods Enzymol., 395:38-48, 2005; Anderson, K L et al, Comparison of rapid methods for the extraction of bacterial DNA from colonic and caecal lumen contents of the pig, J. Appl. Microbiol., 94(6):988-93, 2003; McOrist, A L et al., A comparison of five methods for extraction of bacterial DNA from human faecal samples, J Microbiol Methods, 50(2):131-9, 2002; Yu, Z and Morrison, M, Improved extraction of PCR-quality community DNA from digesta and fecal samples, Biotechniques, 36(5):808-12, 2004; Hume ME et al., Poultry digestive microflora biodiversity as indicated by denaturing gradient gel electrophoresis, Poult Sci., 82(7):1100-7, 2003 and Sharma, R, et al., Extraction of PCR-quality plant and microbial DNA from total rumen contents, Biotechniques, 34(1):92-4, 96-7, 2003.

Optionally, particular microorganisms are selected for during an amplification step such that an amplified culture is enriched in a particular microorganism compared to the sample obtained from the source animal.

A sample of microorganisms obtained from the digestive system of a source animal is a complex mixture of microorganisms. Among the microorganisms in the sample may be a bacterium, a protozoan, a yeast, a fungus, a bacterial spore, a protozoal spore, a yeast spore, a fungal spore, or combinations of these. Further diverse species of these organisms are present in the digestive system of the animal from which the sample is taken. Thus, in one embodiment, diverse species of microorganisms are included in an inventive composition. In a preferred embodiment, more than one species of microorganism is included in an inventive composition. In a further preferred embodiment, 2-4 species of microorganism are included, and more preferably, 5 or more species of microorganism are included in an inventive composition.

In a highly preferred embodiment, at least 50% of the total number of species represented in a sample taken from the digestive system of the source animals are included in a composition according to the invention. Further preferred is an embodiment in which at least 75% of the total number of species represented in a sample taken from the digestive system of the source animals are included in a composition according to the invention. Additionally preferred is an embodiment in which at least 85% of the total number of species represented in a sample taken from the digestive system of the source animals are included in a composition according to the invention. Also preferred is an embodiment in which at least 85-100% of the total number of species represented in a sample taken from the digestive system of the source animals are included in a composition according to the invention.

Among the microorganisms included in an inventive composition may be a bacterium, a protozoan, a yeast, a fungus, a bacterial spore, a protozoal spore, a yeast spore, a fungal spore, or combinations of these.

Food Compositions

A food composition according to an embodiment of the present invention is intended to provide a nutritional benefit to a recipient animal. In particular, an embodiment of an inventive food composition is intended to provide a nutritional benefit to a recipient animal that would historically have been obtained by ingesting at least a portion of the contents of the digestive tract of a source animal. Thus, an embodiment of an inventive food composition includes a digestion and/or fermentation product of a food component of the natural diet of the source animal.

An inventive food composition includes a digestion and/or fermentation product of a food component of the natural diet of the source animal.

In a preferred embodiment, an inventive food composition includes microorganisms typically found in the digestive tract of the source animal and a digestion and/or fermentation product of a food component of the natural diet of the source animal.

Optionally, and preferably, a food composition according to the present invention contains little or substantially none of a food component of the natural diet of the source animal which is undigested or unfermented. Thus, for example, a food composition according to the present invention contains an amount a food component of the natural diet of the source animal which is undigested or unfermented in the range of about 0-5 percent of the total weight of the composition. In further embodiments, this amount is in the range of about 0-2 percent of the total weight of the composition. Preferred is a composition which is substantially free of an undigested or unfermented food component of the natural diet of the source animal.

In further preferred embodiments, an inventive food composition includes one or more digestive enzymes of the source animal, one or more non-enzymatic digestive components such as non-enzymatic digestive secretions of the source animal, as well as other components typically found in the intestinal contents of a source animal.

A food component of the natural diet of the herbivore source animal may be fermented by the action of microorganisms in the digestive system of the source animal and serves as a nutritive medium for such microorganisms. In addition, products of such fermentation, and digestion of the microorganisms themselves serve as a source of nutrition for the source animal.

Many food components of the natural diet of a carnivore or omnivore source animal are digested by the action of enzymes and non-enzymatic components of the digestive system of the source animal. In addition, some of the food components may be fermented by the action of microorganisms in the digestive system of the source animal and serve as a nutritive medium for such microorganisms.

A product of microbial fermentation of a nutritive medium may be included in a food composition. Exemplary products of microbial fermentation of a cellulose-rich nutritive medium, such as grass, include short chain fatty acids such as acetic acid, propionic acid and butyric acid among others.

Digestion products of foods may also be included in an inventive food composition. Exemplary digestion products include peptides, amino acids, oligosaccharides, disaccharides, monosaccharides, glycerol and fatty acids.

Where a human is an intended recipient of an inventive food composition, an exemplary source animal is a weaned cow fed a natural diet over the span of its life. Thus, an inventive food composition includes digestion and/or fermentation products of a food component of the natural diet of such a cow. Preferably also included is an isolated sample of microorganisms, and/or an isolated amplified sample of such microorganisms, from the digestive tract of such a cow. Optionally further included are digestive enzymes and/or non-enzymatic digestive components, as well as other components typically found in the intestinal contents of such a cow.

Where a pet cat is an intended recipient of an inventive food composition, an exemplary source animal is a weaned rodent fed a natural diet over the span of its life. Thus, an inventive food composition includes digestion and/or fermentation products of a food component of the natural diet of such a rodent. Preferably also included is an isolated sample of microorganisms, and/or an isolated amplified sample of such microorganisms, from the digestive tract of such a rodent. Optionally further included are digestive enzymes and/or non-enzymatic digestive components, as well as other components typically found in the intestinal contents of such a rodent.

Where a pet dog is an intended recipient of an inventive food composition, an exemplary source animal is a weaned rabbit fed a natural diet over the span of its life. Thus, an inventive food composition includes digestion and/or fermentation products of a food component of the natural diet of such a rabbit. Preferably also included is an isolated sample of microorganisms, and/or an isolated amplified sample of such microorganisms, from the digestive tract of such a rabbit. Optionally further included are digestive enzymes and/or non-enzymatic digestive components, as well as other components typically found in the intestinal contents of such a rabbit.

Optionally, a substantial portion of the microorganisms are separated from the product of microbial fermentation in an inventive food composition. Where present, a substantial portion of microorganisms are preferably living when included in a food composition according to the invention. However, optionally, a substantial portion of the microorganisms are killed prior to consumption by a recipient animal.

The source animal is preferably weaned and an animal which has been fed a natural diet over a period of time extending from weaning to a time at which microorganisms are isolated from the digestive system of the source animal.

A composition formulated as a food may further include foods, flavorings, vitamins or other additives which provide nutritive value or other benefit to the consuming animal. Such other benefits include, for example, making the composition palatable.

A composition is provided which is an intermediate in a process for producing a food composition according to the present invention. Such a composition includes, in one embodiment, a plurality of microorganisms isolated from the digestive system of a source animal, a nutritive medium for the plurality of microorganisms and a product of fermentation of the nutritive medium by the microorganisms.

The nutritive medium for the microorganisms included in an embodiment of an inventive food composition is a food consumed by a source animal as part of a natural diet. Optionally, the nutritive medium is substantially indigestible by the second type of animal which is intended to consume the food composition.

In a highly preferred embodiment, the nutritive medium contains a grass and/or ground plant and substantially excludes a cereal grain. A nutritive medium is further preferably organically grown and minimally processed.

Methods Relating to Food Compositions

In one embodiment, a method of preparing an inventive food composition includes securing an animal that has been a traditional source of food for humans and that has been feeding from naturally occurring food in its native habitat and obtaining a fresh raw sample of at least partially digested contents from the animal intestine. Microorganisms from the partially digested contents are used to ferment the naturally occurring animal food, whereby the fermented naturally occurring animal food may be used as a food for use by humans. By way of example, microbacteria from the intestine of a cow can be used to ferment batches of grasses naturally eaten by that breed of cow.

In another embodiment, a method is provided for creating a food for use by humans which includes the steps of securing an animal that has been a traditional source of food for humans and that has been feeding from naturally occurring food in its native habitat and obtaining a fresh raw sample of at least partially digested contents from the animal intestine. Additional steps include extracting microorganisms from the partially digested contents and using the microorganisms to ferment batches of the naturally occurring animal food, whereby the fermented naturally occurring animal food may be used as a food for use by humans.

A method of preparing an inventive food composition according to one embodiment includes contacting a nutritive medium for microorganisms and a plurality of microorganisms to produce a mixture including the nutritive medium and the plurality of microorganisms. The mixture is incubated under conditions suitable for fermentation of the nutritive medium by at least a portion of the microorganisms to produce a fermentation product. Conditions suitable for fermentation of a nutritive medium are achieved by controlling factors such as temperature, oxygen level, volume, osmolality, nutritive medium concentration, product concentration, and pH. In one embodiment, conditions suitable for fermentation are designed to approximate the environment found in the portion of the source animal digestive system from which the microorganisms are extracted.

A process for producing a food composition in one embodiment includes contacting one or more foods typically consumed as a part of a natural diet of a source animal, a plurality of microorganisms isolated from a source animal and, optionally, a component typically found in the digestive system of the source animal, such as an enzyme produced by a cell of the digestive system of a source animal, a non-enzymatic digestive secretion produced by a cell of the digestive system of a source animal, or a combination thereof; to produce a mixture. The mixture is incubated under conditions suitable for reaction of the mixture to produce a fermentation product of the one or more foods and/or a digestion product of the one or more foods. Highly preferred are incubation conditions designed to approximate the environment found in the portion of the source animal digestive system from which the microorganisms are extracted.

Conditions suitable for fermentation and/or digestion which approximate the environment in a particular portion of the digestive system of a source animal are known, for instance, as described in Barnett, A. J. G. and Reid, R. L., Reactions in the Rumen, Edward Arnold Publishers, Ltd., 1961; Stevens, C. E. and Hume, I. D., Comparative Physiology of the Vertebrate Digestive System, 2^{nd} Ed., Cambridge University Press, 1995; and Dougherty, B. S. et al., Physiology of Digestion in the Ruminant, Butterworth, Inc., Washington, 1965. For example, such conditions include a temperature close to the body temperature of the source animal, in the range of about 35-43 degrees Centigrade. Such conditions further include a pH in the range of about pH 1-pH 8, depending on the portion of the digestive system concerned. Fermentation and/or digestion may be performed in volumes approximating the volume of a portion of the digestive system of a source animal or may be scaled up or down where desired.

In one embodiment, atmospheric conditions suitable for fermentation are generally low oxygen or anaerobic conditions, typically in an atmosphere containing less than 10% oxygen.

A mixture incubated under conditions suitable for fermentation and/or digestion to produce a fermentation and/or digestion product optionally includes further components promoting production of a fermentation and/or digestion product. For example, one or more salts may be added to adjust the ionic composition of the mixture.

In one embodiment, such further components are included so as to closely approximate the conditions present in the digestive system of the source animal in composition and concentration of the incubated mixture. For example, enzymes produced by cells of the digestive system of the source animal may be included. Such enzymes include lipases, poly- and oligo-saccharidases, proteases, peptidases and polynucleotide nucleases and oligonucleotide nucleases. Further, non-enzymatic digestive secretions such as bile, or components of bile, such as bile salts, may be included.

Digestive enzymes and non-enzymatic digestive secretions may be isolated from a source animal. Alternatively, digestive enzymes and/or non-enzymatic digestive secretions may be produced recombinantly or by chemical synthesis.

A mixture incubated under conditions suitable for fermentation and/or digestion to produce a fermentation and/or digestion product may further be subjected to movement to approximate the mixing and movement of intestinal contents in situ. Such movement may include gentle stirring or rocking of a container holding a mixture.

Fermentation and/or digestion reactions may be performed in batches or continuously. For instance, an incubation container having an inlet and an outlet is provided so that a food component of the natural diet of a source animal, microorganisms, gases and other components may be added through the inlet, incubated therein, and fermentation and/or digestion products, microorganisms, gases and other components may be removed via the outlet.

Fermentation and/or digestion reactions of a mixture may be performed for a time sufficient to ferment and/or digest the mixture such that substantially all of the food component is fermented and/or digested to produce a resulting food composition. Alternatively, an unfermented and/or undigested food component may be removed such that to produce a food composition substantially free of an unfermented and/or undigested food component. For example, unfermented and/or undigested food may be separated out by centrifugation.

Optionally, a substantial portion of the microorganisms is removed from the mixture following fermentation and/or digestion. Removal may be effected by physical separation, such as by centrifugation, and/or by killing the microorganisms, such as by lysis for example.

Any patents or publications mentioned in this specification are incorporated herein by reference to the same extent as if each individual publication was specifically and individually indicated to be incorporated by reference.

Methods and compositions described herein are presently representative of preferred embodiments. Thus, they are exemplary and are not intended as limitations on the scope of the invention or inventions. Changes therein and other uses will occur to those skilled in the art. Such changes and other uses are encompassed within the spirit of the invention as defined by the scope of the claims.

I claim:

1. A food composition, comprising:
a digestion or fermentation product of a food component of the natural diet of a source animal having a native habitat, wherein the source animal is a traditional food source of a second type of animal.

2. The food composition of claim 1 wherein the food component comprises a grass.

3. The food composition of claim 1, further comprising a plurality of microorganisms isolated from a source animal.

4. The food composition of claim 1 wherein the second type of animal is a human.

5. The food composition of claim 1 wherein the source animal is a weaned ruminant.

6. The food composition of claim 1 wherein the native habitat is Africa.

7. The food composition of claim 5 wherein the weaned ruminant is selected from the group consisting of: a cow, a sheep, a goat, a bison, a buffalo, a cape buffalo, a deer, an elk, an antelope, a moose, and a llama.

8. The food composition of claim 1 wherein the source animal is selected from the group consisting of: a pig, a chicken, a turkey, a game bird, a fish, a shellfish, a horse, a rodent, a rabbit, and a hare.

9. The food composition of claim 1 wherein the source animal has been fed a natural diet over a period of time extending from weaning to a time at which microorganisms are isolated from the digestive system of the source animal.

10. The food composition of claim 1 wherein the second type of animal is an animal commonly kept as a household pet.

11. The food composition of claim 10 wherein the source animal is selected from the group consisting of: a ruminant, a pig, a poultry animal, a bird, a fish, a rodent, a rabbit, a hare, a reptile and an amphibian.

12. The food composition of claim 1, further comprising:
a digestive enzyme typically found in the digestive system of the source animal.

13. The food composition of claim 1, further comprising:
a non-enzymatic secretion of the digestive system of the source animal.

14. A process for producing a food composition, comprising:
contacting one or more foods typically consumed as a part of a natural diet of a source animal and a plurality of microorganisms isolated from the source animal to produce a mixture; and
incubating the mixture under conditions suitable for reaction of the mixture to produce a product selected from the group consisting of: a fermentation product of the one or more foods, a digestion product of the one or more foods, and a combination thereof.

15. The process of claim 14 further comprising:
adding to the mixture a component typically found in the digestive system of the source animal, the component selected from the group consisting of: an enzyme produced by a cell of the digestive system of the source animal, a non-enzymatic digestive secretion produced by a cell of the digestive system of the source animal, and a combination thereof.

16. A composition, comprising:
a food component of the natural diet of a source animal having a native habitat, wherein the source animal is a traditional food source of a second type of animal; a plurality of microorganisms isolated from a source animal; and a digestion or fermentation product of the food component.

17. The composition of claim 16 wherein the food component is a grass.

18. The composition of claim 16 wherein the source animal is a weaned ruminant.

19. The composition of claim 16 wherein the second type of animal is a human.

20. The composition of claim 16 wherein the second type of animal is an animal commonly kept by a human as a household pet.
